(19) United States
(12) Patent Application Publication
(10) Pub. No.: US 2023/0381369 A1
(43) Pub. Date: Nov. 30, 2023

(54) REUSABLE, WATER-RESISTANT, ADHERABLE TAPE AND USE THEREOF

(71) Applicant: Abeego Designs Inc., Victoria (CA)

(72) Inventor: Toni Marie Desrosiers, Victoria (CA)

(73) Assignee: Abeego Designs Inc., Victoria, BC (CA)

(21) Appl. No.: 18/320,787

(22) Filed: May 19, 2023

(30) Foreign Application Priority Data
May 25, 2022 (CA) 3160180

(51) Int. Cl. A61L 15/52; C08J 5/24; C08L 93/00; A61F 17/00; A61L 15/44; A61L 15/22; A61L 15/20; A61L 15/42

(52) U.S. Cl. CPC A61L 15/52; C08J 5/245; C08L 93/00; A61F 17/00; A61L 15/44; A61L 15/225; A61L 15/20; A61L 15/425; C08J 2393/00; C08J 2491/06

(57) ABSTRACT

A tape is provided which is adherable in the absence of an adhesive layer and is water-resistant, the tape comprising a fabric layer which includes a first side and a second side and a mixture of beeswax, tree resin and jojoba oil, the mixture infusing the fabric layer and forming a first outer layer on the first side and a second outer layer on the second side, wherein the tape is between about a half inch to four inches in width and can be cut to length.

### CROSS REFERENCE TO RELATED APPLICATIONS

The present invention is related to, and claims the benefit of, Canadian Patent Application Serial No. 3160180, filed on May 25, 2022, and entitled REUSABLE, WATER-RESISTANT, ADHERABLE TAPE AND USE THEREOF and which is hereby incorporated in its entirety including all tables, figures, and claims.

### FIELD

The present technology is directed towards a multi-purpose length of low-tack tape. More specifically, it is a double-sided material, provided as a roll of tape that will grip a surface or adhere to itself but does not include adhesive layers and therefore does not leave a sticky residue when removed.

### BACKGROUND

United States Patent Application Publication No. 20220127501 discloses a high-performance ultra-thin double-sided adhesive tape, including a polyester film substrate and a high-performance adhesive coating coated on both sides of the polyester film. The high-performance adhesive coating includes the following parts by weight of each component uniformly mixed: 100-140 parts by weight of high-performance acrylic pressure sensitive adhesive, 15-20 parts by weight of curing agent, and 130-160 parts by weight of ethyl acetate. The thickness of the adhesive tape is 0.003-0.010 mm, with a good adhesion and a mechanical strength. The tape leaves a sticky residue when removed and is not water-resistant. It is not reusable.

United States Patent Application Publication No. 20220098448 discloses adhesive tapes that comprise a textile carrier and a pressure-sensitive adhesive, applied on at least one side of the carrier, in the form of a thickened dried polymer dispersion, wherein the unthickened dried polymer dispersion comprises: (a) 30.0 to 98.0 wt % of monomeric acrylates; (b) 0 to 50.0 wt % of ethylenically unsaturated comonomers which are not acrylates; (c) 1.0 to 10.0 wt % of tackifier; and (d) 1.0 to 10.0 wt % of kaolin, wherein a rheological additive is added to the polymer dispersion so that the polymer dispersion has a viscosity before drying of 40 to 100 Pa*s at a shear rate of 10/s and a viscosity of 3000 to 8000 Pa*s at a shear rate of 0.01/s. The tape leaves a sticky residue when removed and is not water-resistant. It is not reusable.

United States Patent Application Publication No. 20220041902 discloses powerful adhesive tapes, which rapidly coat rough and/or curved surfaces with different surface energies and thereby build up high adhesion. The adhesive joints formed in this way furthermore have good shear strength, even at elevated temperatures. Finally, enduring mechanical loading on the adhesive joints does not lead to the adhesive tape peeling from the surface. This is achieved with an adhesive tape comprising at least one foamed layer and at least one adhesive compound layer, wherein the adhesive compound layer contains at least one poly(meth)acrylate, and the poly(meth)acrylate is cross-linked with at least one covalent and at least one coordinative cross-linker. The tape leaves a sticky residue when removed and is not water-resistant. It is not reusable.

United States Patent Application Publication No. 20210340410 discloses a weather proof pressure sensitive adhesive tape composition having 15 years or more dwell time in ambient conditions, comprising ambient humidity in the range of RH 30% to 100% RH including aggressive under water conditions, ambient pressures in the range of 0.75 bar to 1.5 bar at normal temperature, ambient temperature in the range of (−) 30 .degree. C. to 75 .degree. C. at normal pressure, ambient acidity and alkalinity in the range of pH 4.5 to pH 10.5 in water, ambient salinity of water in under water conditions of up to 1.2 mole equivalent of sodium chloride and ambient surface fluid resistance of up to 2.7 .times.10-3 cubic meter per second per meter contact edge of the tape. The tape leaves a sticky residue when removed. It is not reusable.

United States Patent Application Publication No. 20130269861 discloses an adhesive tape intended more particularly for wrapping cables, comprising a textile backing and, coated on at least one side thereof, an adhesive comprising at least one vinyl aromatic block copolymer and an at least partly hydrogenated tackifier resin. The tape leaves a sticky residue when removed and is not water-resistant. It is not reusable.

United States Patent Application Publication No. 20120279755 discloses an adhesive tape that has a preferably textile carrier and of a pressure-sensitive adhesive which is applied on at least one side of the carrier and is in the form of a dried polymer dispersion. The polymer being synthesized from (a) 5% to 25% by weight, preferably 10% to 22% by weight, of ethylene, (b) 30% to 69% by weight, preferably 40% to 60% by weight, of alkyl acrylic ester having C.sub.4 to C.sub.12 alkyl radicals, (c) 20% to 55% by weight, preferably 28% to 38% by weight, of vinyl acetate, (d) 0% to 10% by weight of other ethylenically unsaturated compounds, and the pressure-sensitive adhesive comprising between 15 and 100 parts by weight of a tackifier (based on the mass of the dried polymer dispersion). The tape leaves a sticky residue when removed and is not water-resistant. It is not reusable nor is it a natural product.

United States Patent Application Publication No. 20090145539 discloses a removable adhesive tape comprising a highly extensible and substantially inelastic backing and a layer of pressure-sensitive adhesive. The backing has a high tensile strength, has a lengthwise elongation at break of at least about 150% with less than about 50% elastic recovery after stretching. The adhesive can be a normally tacky and pressure-sensitive adhesive and is coated on at least one surface of the film backing. The adhesive is preferably highly extensible, does not separate from the backing during stretching, and has higher cohesion than adhesion to any suitable substrate. After being applied to a substrate, the adhesive tape of the present invention becomes firmly bonded, but can be easily removed without damaging the substrate by simply stretching it in a direction substantially parallel, i.e., less than about 35 .degree, to the surface of the substrate. The tape leaves a sticky residue when removed and is not water-resistant. Although it can be removed, it is not reusable.

United States Patent Application Publication No. 20070184736 discloses adhesive tape having a backing material applied to at least one side of which is an adhesive and whose strength in lengthwise direction is reinforced by integrated or added fibers or filaments, characterized in that the adhesive tape on at least one long edge has incisions, the incisions severing per long edge at least one filament aligned substantially parallel to the cut edge. The tape leaves a sticky residue when removed and is not water-resistant. It is not reusable.

United States Patent Application Publication No. 20060240208 discloses a waterproof and airtight double-sided pressure-sensitive adhesive that tape includes a substrate and a pressure-sensitive adhesive layer on the both surfaces thereof, wherein the substrate is a rubber-made sheet; and the surface of at least one of the pressure-sensitive adhesive layers is protected by a release liner having elongation properties. It is preferable that the rubber-made sheet as the substrate has an elongation at the time of breakage (distance between two gage marks: 40 mm, drawing rate: 300 mm/min) of from 200 to 1,200% in both the longitudinal direction and the cross direction and a stress under 100% elongation (distance between two gage marks: 40 mm, drawing rate: 50 mm/min) of from 10 to 100 N/cm.sup.2 in both the longitudinal direction and the cross direction. The tape leaves a sticky residue when removed. It is not reusable.

United States Patent Application Publication No. 20200164599 discloses a relatively thick, rubberized adhesive tape that is applied to an article to repair, patch, bond, seal, or waterproof the article, to join two or more articles, and/or to modify the appearance and/or physical properties of the article, such as by thermal insulation, shock resistance, vibration resistance, electrical insulation, and the like. The tape leaves a sticky residue when removed. It is not reusable.

United States Patent Application Publication No. 20140190623 discloses a protective wrap for application to a wooden baseball bat to prevent the release of fragments should the baseball bat shatter during use is formed of an elongated piece of polymeric film that has a first end and an opposite second end, a first side edge, and an opposite second side edge. The piece has a first tapered region terminating at the first end and a second tapered region terminating in the second end. Within each of the first and second tapered regions, the first and second side edges converge. The piece also includes a main section located between the first and second tapered regions. The first and second side edges are parallel to one another along the length of the main section. A back surface of the polymeric film includes an adhesive for adhering the polymeric film to the baseball bat and to any underlying polymeric film.

What is needed is a reusable material that is provided as a grippy tape that does not leave a sticky residue when removed. It would be preferable if it was double sided. It would be preferable if the tape gripped a surface or adhered to itself, while not including adhesive layers. It would be preferable if the tape was water-resistant. It would be preferable if the tape could be used to protect surfaces, to repair items, to provide a grip surface and to attach items to one another. It would be preferable if the tape could be provided as a roll of tape. It would be preferable if it was breathable and was anti-microbial.

### SUMMARY

The present technology is a reusable material that is provided as a grippy tape that does not leave a sticky residue when removed. The tape is double sided. The tape grips a surface or adheres to itself, while not including adhesive layers. The tape is water-resistant. The tape can be used to protect surfaces, to repair items, to provide a grip surface and to attach items to one another. The tape can be in pre-cut lengths or as a roll of tape. It is breathable and is anti-microbial.

In one embodiment, a tape is provided which is adherable in the absence of an adhesive layer, is breathable, is reusable and is water-resistant, the tape comprising a fabric layer which includes a first side and a second side and a mixture of a natural wax, a tree resin and a plant oil, the mixture infusing the fabric layer and forming a first outer layer on the first side and a second outer layer on the second side, wherein the tape is between about a half inch to four inches in width and can be cut to length.

In the tape, the natural wax may be beeswax and the plant oil is jojoba oil.

In the tape, the fabric layer may be a muslin layer.

In the tape, the mixture may be about 35% to about 60% of the weight of the tape.

In the tape, the mixture may comprise about 30% to about 50% by weight beeswax, about 9% to about 24% by weight tree resin and about 3% to about 10% by weight jojoba oil.

In the tape, the tree resin may be Damar resin.

In another embodiment, a safety kit is provided, the safety kit comprising a tape which is adherable in the absence of an adhesive layer, is anti-microbial, is breathable and is water-resistant, the tape comprising a fabric layer which includes a first side and a second side and a mixture of a natural wax, a tree resin and a plant oil, the mixture infusing the fabric layer and forming a first outer layer on the first side and a second outer layer on the second side, a pair of scissors and a pack of sterile gauze.

In the safety kit, the fabric layer may be a muslin layer.

In the safety kit, the mixture may be about 35% to about 60% of the weight of the tape.

In the safety kit, the mixture may comprise about 30% to about 50% by weight beeswax, about 9% to about 24% by weight tree resin and about 3% to about 10% by weight jojoba oil.

In the safety kit, the tree resin may be Damar resin.

In the safety kit, the tape may be between about a half inch to about four inches in width and can be cut to length with the scissors.

In another embodiment, a water-resistant bandage is provided, the water-resistant bandage comprising a tape which is adherable in the absence of an adhesive layer, is anti-microbial, is breathable and is water-resistant, the tape comprising a fabric layer which includes a first side and a second side and a mixture of a natural wax, a tree resin and a plant oil, the mixture infusing the fabric layer and forming a first outer layer on the first side and a second outer layer on the second side, and a gauze which is positioned on the tape such as at least a perimeter of the tape extends beyond the gauze.

In the water-resistant bandage, the fabric layer may be a muslin layer.

In the water-resistant bandage, the mixture may be about 35% to about 60% of the weight of the tape.

In the water-resistant bandage, the mixture may comprise about 30% to about 50% by weight beeswax, about 9% to about 24% by weight tree resin and about 3% to about 10% by weight jojoba oil.

In the water-resistant bandage, the tree resin may be Damar resin.

In the water-resistant bandage, the tape may be between about a quarter of an inch to about four inches in width.

In another embodiment, a method of sealing an opening in an article is provided, the method comprising: selecting a tape which is adherable in the absence of an adhesive layer and is water-resistant, the tape comprising a fabric layer which includes a first side and a second side and a mixture of a natural wax, a tree resin and a plant oil, the mixture infusing the fabric layer and forming a first outer layer on the first side and a second outer layer on the second side; cutting the tape to a desired length; and pressing the tape on the article such that the opening is occluded and the tape is wrapped about itself, thereby sealing the opening.

In another embodiment, a hand grip tape is provided for use on sporting equipment, wherein the hand grip tape is adherable in the absence of an adhesive layer, is breathable, is anti-microbial, is reusable and is water-resistant, the tape comprising a fabric layer which includes a first side and a second side and a mixture of a natural wax, a tree resin and a plant oil, the mixture infusing the fabric layer and forming a first outer layer on the first side and a second outer layer on the second side, wherein the tape can be cut to length.

In another embodiment, a method of preparing a hand grip on a piece of equipment which includes a grip zone is provided, the method comprising selecting a grip tape that is adherable in the absence of an adhesive layer, is breathable, is anti-microbial, is reusable and is water-resistant, the tape comprising a fabric layer which includes a first side and a second side and a mixture of a natural wax, a tree resin and a plant oil, the mixture infusing the fabric layer and forming a first outer layer on the first side and a second outer layer on the second side; and wrapping the grip zone such that at least a portion of a width of the grip tape is overlapping itself.

### BRIEF DESCRIPTION OF DRAWINGS

FIG. 1 is a schematic of the roll of tape of the present technology.

FIGS. 2A and 2B are side views of a piece of tape.

FIG. 3 is block diagram showing the steps in manufacturing the tape.

FIG. 4A-H are schematics of the tape in use.

FIG. 5 is a schematic of an emergency kit.

### DESCRIPTION

Except as otherwise expressly provided, the following rules of interpretation apply to this specification (written description and claims): (a) all words used herein shall be construed to be of such gender or number (singular or plural) as the circumstances require; (b) the singular terms “a”, “an”, and “the”, as used in the specification and the appended claims include plural references unless the context clearly dictates otherwise; (c) the antecedent term “about” applied to a recited range or value denotes an approximation within the deviation in the range or value known or expected in the art from the measurements method; (d) the words “herein”, “hereby”, “hereof”, “hereto”, “hereinbefore”, and “hereinafter”, and words of similar import, refer to this specification in its entirety and not to any particular paragraph, claim or other subdivision, unless otherwise specified; (e) descriptive headings are for convenience only and shall not control or affect the meaning or construction of any part of the specification; and (f) “or” and “any” are not exclusive and “include” and “including” are not limiting. Further, the terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. Where a specific range of values is provided, it is understood that each intervening value, to the tenth of the unit of the lower limit unless the context clearly dictates otherwise, between the upper and lower limit of that range and any other stated or intervening value in that stated range, is included therein. All smaller sub ranges are also included. The upper and lower limits of these smaller ranges are also included therein, subject to any specifically excluded limit in the stated range.

Unless defined otherwise, all technical and scientific terms used herein have the same meaning as commonly understood by one of ordinary skill in the relevant art. Although any methods and materials similar or equivalent to those described herein can also be used, the acceptable methods and materials are now described.

### Definitions

Natural wax—in the context of the present technology, a natural wax is a wax that is derived from plants, animals or insects. It may be derived from a single source or may be a mixture of natural waxes.

Beeswax—The main components are palmitate, palmitoleate, and oleate esters of long-chain (30-32 carbons) aliphatic alcohols, with the ratio of triacontanyl palmitate CH3(CH2)29O—CO—(CH2)14CH3 to cerotic acid CH3(CH2)24COOH, the two principal components, being 6:1. Beeswax can be classified generally into European and Oriental types. The saponification value is lower (3-5) for European beeswax, and higher (8-9) for Oriental types.

Beeswax has a relatively low melting point range of 62 to 64° C. (144 to 147° F.). If beeswax is heated above 85° C. (185° F.) discoloration occurs. The flash point of beeswax is about 204.4° C. (400 to 470° F.). Density at 15° C. is 958 to 970 kg/m³.

Natural beeswax—When cold it is brittle; at ordinary temperatures it is tenacious; its fracture is dry and granular. The sp. gr. at 15° C. is from 0.958 to 0.975, that of melted wax at 98°-99° compared with water at 15.5° is 0.822. It softens when held in the hand, and melts at 62°-66°; it solidifies at 60.5°-63° C.

Fabric—in the context of the present technology, a fabric includes knits and weaves of fibres such as, but not limited to nylon, rayon, carbon fibre, silk, mechanically processed bamboo (bamboo linen), bamboo, cotton, hemp, linen, sisal, hardwood fibre, softwood fibre and mixtures thereof.

Natural fabric—in the context of the present technology, natural fabric includes knits and weaves of silk and of plant derived fibres, such as, but not limited to cotton, jute, kenaf, hemp, linen, sisal, hardwood fibre, mechanically processed bamboo (bamboo linen), softwood fibre, herbaceous dicot fibres and monocot fibres and mixtures thereof. Natural fabric does not include fabrics from regenerated fibres.

Plant oil—in the context of the present technology, a plant oil is an oil that is derived from plants, for example, but not limited to canola oil, rapeseed oil, jojoba oil, olive oil, safflower oil, corn oil and palm oil.

Jojoba oil—This is a liquid wax ester from seeds of the seed of the Simmondsia chinensis plant. It has not been saponified. The melting point of jojoba oil is approximately 10° C. and the iodine value is approximately 80. It has a viscosity of 48 cSt at 99° C. and 127 cSt at 37.8° C. it is composed largely of 11-Eicosenoic Acid (C20:1) [30.3%], Docosenoic Acid (C22:1) [14.2%], Docosdienoic acid (C22:2) [33.7%] and 9-Godoelic Acid (C20:1) [14.6%]. There are no trigycerides. Other oils sharing the characteristics of jojoba oil are suitable substitutes, with viscosity, melting point and a high percentage of long, relatively straight chain fatty acids, with one or two double bonds and a lack of trigycerides being the more important characteristics for the present technology.

Tree resin—The preferred resin is from the Damar tree and is referred to also as Damar gum. Without being bound to theory, the tree resin increases the melting point of the wax and adds to the adhesive qualities when mixed with the oil. The damar crystals melt at about 225° C. The resin is obtained from the Dipterocarpaceae family, principally those of the genera Shorea, Balanocarpus or Hopea. Other resins or exudates from plants and trees that provide the desired traits can be used wholly or in part as substitutes, for example, resin from the copal tree, Protium copal (Burseraceae) or the mastic tree, Pistacia lentiscus or sandarac from the Tetraclinis articulata tree may substitute for the Damar tree. Accordingly, the term “tree resin” refers to any of the above-mentioned resins or combinations thereof. Up to about 5% candelilla or carnauba wax may be included in any of the resins or mixtures thereof.

Grip zone—in the context of the present technology a grip zone is where a user holds the equipment with their hands. The grip zone is for example, but not limited to, a bike handlebar, a hockey stick shaft, a golf club shaft, a chin-up bar, a paint roller pole and a kayak paddle shaft.

### DETAILED DESCRIPTION

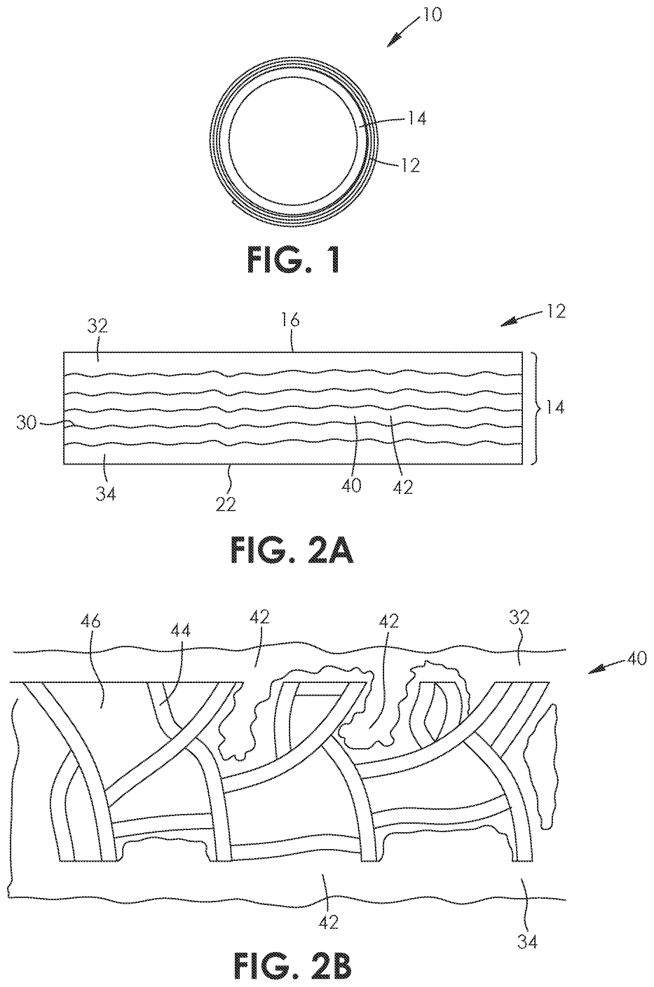

A roll of tape, generally referred to as 10 is shown in FIG. 1. The tape 12 is wrapped around a core 14. As shown in FIG. 2A, the tape 12 has an edge 14, a first surface 16 and a second surface 22. The edge 14 has an inner layer 30, a first outer layer 32, and a second outer layer 34. The inner layer 30 includes a planar substrate 40 and a formulation 42. As shown in FIG. 2B, the formulation 42 may sandwich, coat, impregnate, infiltrate, partially coat, cover, partially cover, or infuse the fibres 44 of the fabric 40. It substantially fills the interstitial spaces 46. The formulation 42 in the first outer layer 32 and the second outer layer 34 is continuous with the formulation in the interstitial spaces 46. Both the first surface 16 and the second surface 22 are composed in their entirety of the formulation 42. The first and second outer layers 32, 34 need only be thick enough to form the first and second surface 16, 22.

In one embodiment, the formulation 42 is made from one or more natural waxes, one or more plant oils and tree resin.

In another embodiment, the formulation 42 is made from a single natural wax, a single plant oil and a single tree resin.

In the preferred embodiment, the formulation 42 is made of beeswax, Damar resin, and Jojoba oil. A mixture of these components is heated and the substrate is infused and/or impregnated and coated with the mixture. The formulation is applied to the substrate using either wet waxing or dry waxing techniques.

The preferred fabrics are made with natural fibres, such as knits and weaves of, for example, but not limited to cotton, hemp, linen, sisal, silk and mixtures thereof. The preferred fabric weight is about 3.5 ounces to about 5.4 ounces. The thread count is preferably about 100 to about 300.

The formulation 42 is about 35% to about 60% of the weight of the tape, preferably 50%. In a preferred embodiment, the fabric is muslin. The tape 12 is about 0.30 mm thick with the first outer layer 32 being about 0.05 mm thick and the second outer layer being about 0.05 mm thick and the inner layer 30 being about 0.25 mm thick. Thus, the inner layer 30 comprises at least about 75% of the thickness of the tape 12 and not more than about 90% of the thickness of the tape 12. The formulation is about 30% to about 50% tree resin, and about 50% to about 70% beeswax, preferably about 30% tree resin, and about 70% beeswax. It is preferred that the tree resin is Damar resin. In another embodiment, the mixture comprises about 30% to about 50% by weight beeswax, about 9% to about 24% by weight tree resin and about 3% to about 10% by weight jojoba oil.

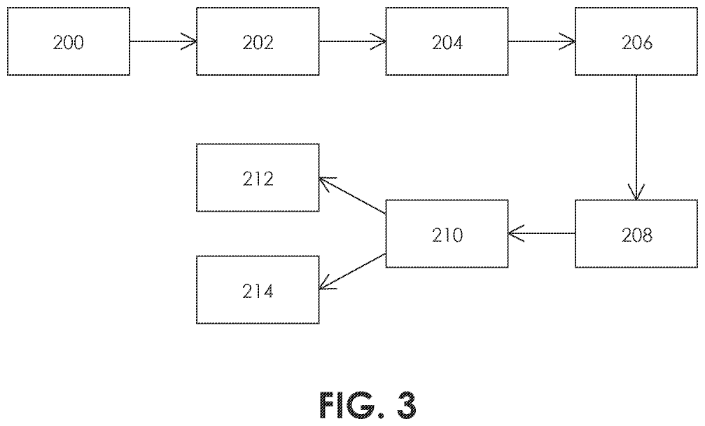

The method of producing the tape 12 is shown in FIG. 3. The components of the formulation are weighted 200, placed in a container 202, heated 204 until the components have melted, and mixed 206. The fabric layer is then fed 208 through the molten mixture and is allowed to cool 210 before being rolled 212 on to the core 14. Alternatively, the tape 12 is cut 214 into predetermined lengths. The tape 12 is reusable and is water-resistant.

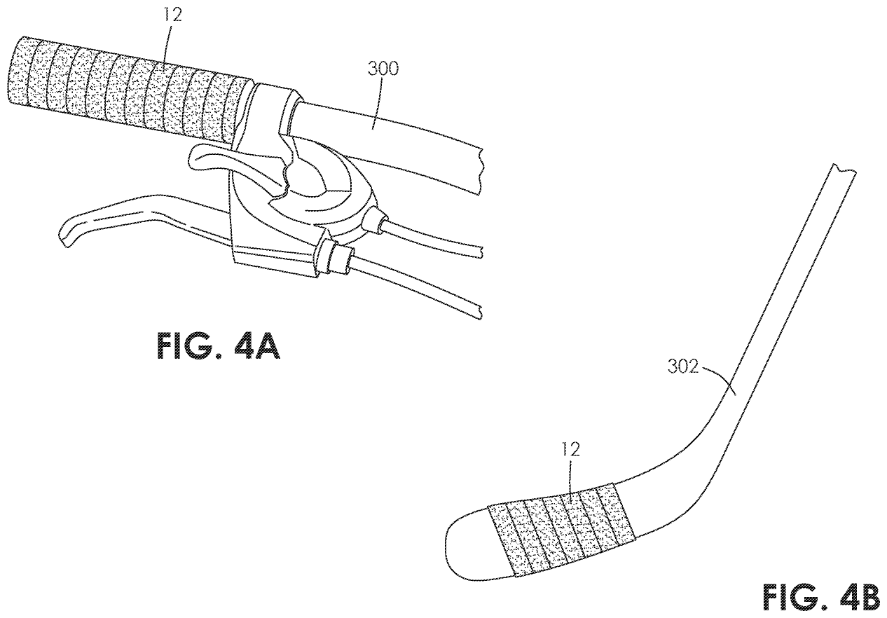

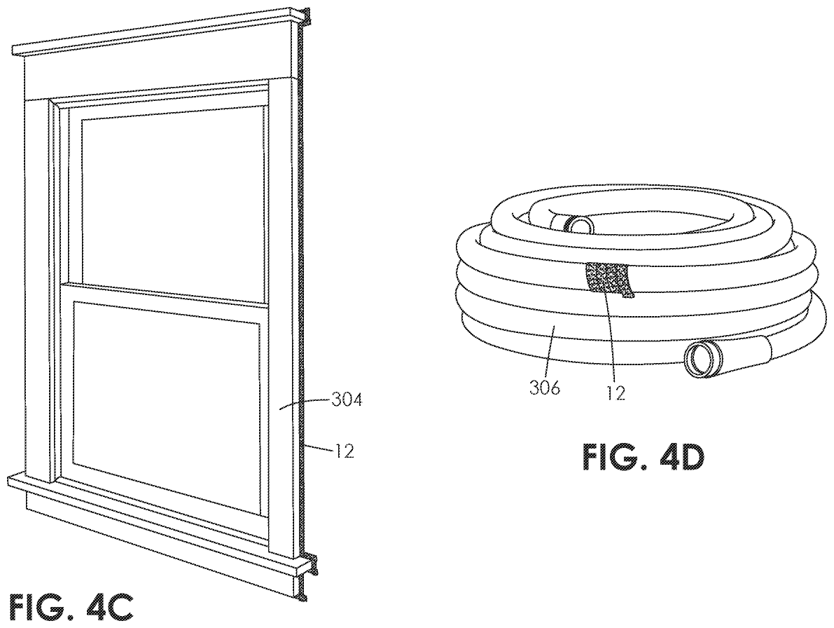

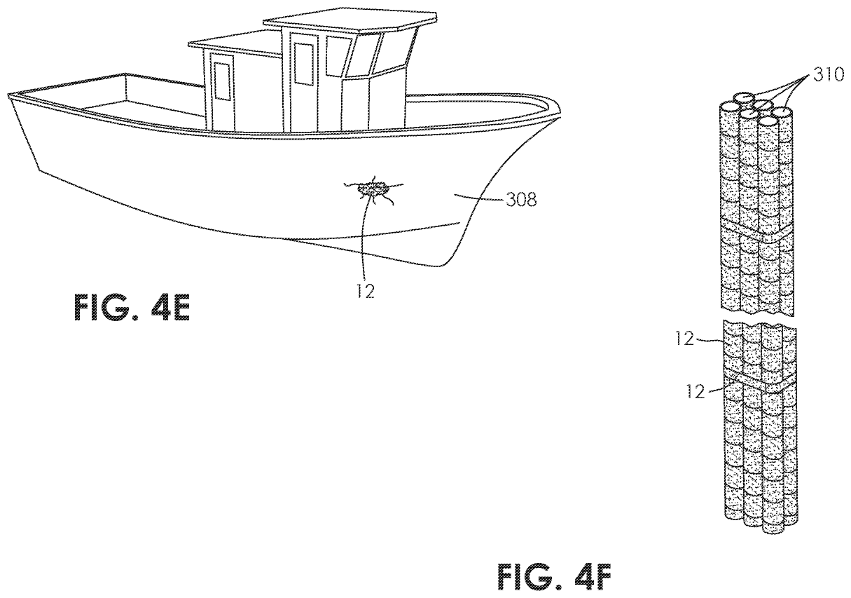

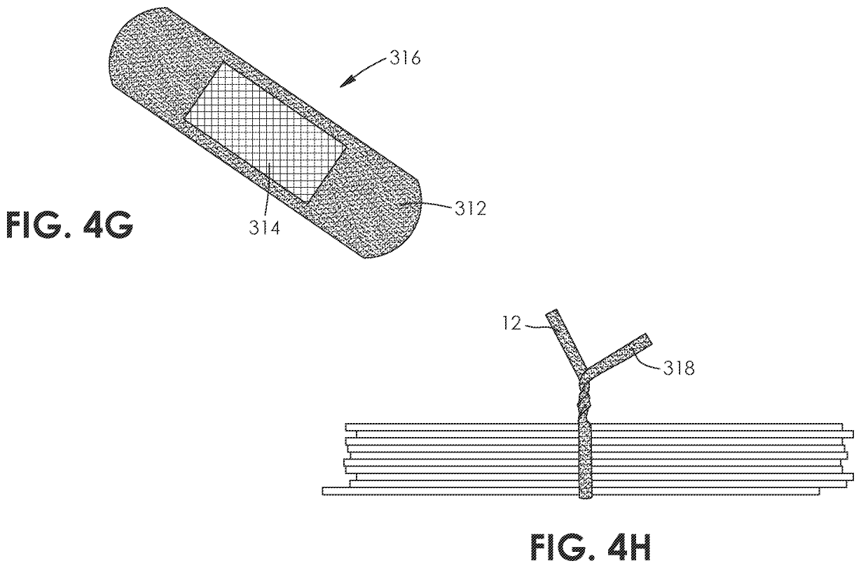

As shown in FIG. 4A-H, there are numerous uses for the tape 12. FIG. 4A shows a combination of a road bike handlebar 300 and the tape 12. The tape 12 is acting as a grip tape. FIG. 4B shows a combination of a hockey stick 302 and the tape 12. Advantageously, the grip tape is reusable, does not leave a sticky residue and is anti-microbial. The tape 12 can be used as a grip tape in many applications. FIG. 4C shows the tape 12 being used to mask a window 304 in preparation for painting the sill. FIG. 4D shows a combination of a garden hose 306 and the tape 12, wherein the tape 12 is wrapped around the hose, adhering to itself in order to seal the leak. As would be known to one skilled in the art, if the tape 12 can be used to seal a hole in a hose it similarly can be used to seal any leak by wrapping around the article and adhering to itself. FIG. 4E shows a combination of a boat hull 308 with a crack, wherein the tape 12 is twisted and packed into the crack, much like oakum would be used. FIG. 4F shows a combination of the tape 12 and a bundle of pipes 310, wherein the pipes 310 are separated from one another with a layer of the tape 12 and are then bound together with the tape 12. As shown in FIG. 4G, the tape 12 is combined with gauze 314 and is used as a bandage, generally referred to as 316. The bandage 316 is breathable and water-resistant and the tape 12 is anti-microbial. As shown in FIG. 4H, the tape 12 can be used as a twist tie 318. The tape 12 leaves no sticky residue as it is adherable while lacking an adhesive layer. Advantageously, the tape 12 is anti-microbial and is malleable.

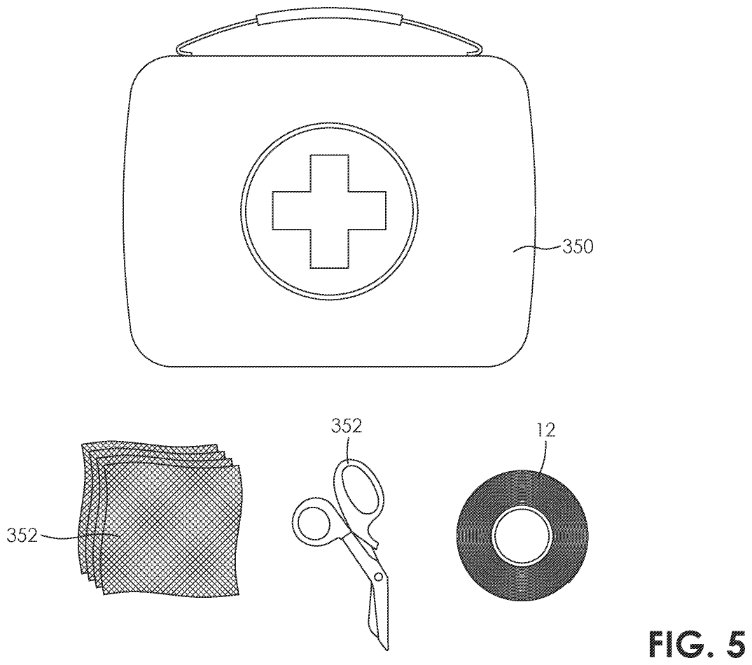

As shown in FIG. 5, the tape 12 is provided as an emergency kit, generally referred to as 350. The emergency kit 350 includes a pair of scissors 352, a sterile pack of gauze 354 and the tape 12. The tape 12 can then be used as a protective layer, as a grip tape, as a sealant, as part of a bandage, and the like.

The fact that the tape 12 can be reused and is preferably made with a natural fabric makes the tape 12 a green alternative to traditional sealants, bandages, ties, grip tapes, double sided tapes, masking tapes and the like. It will grip a surface and will adhere to itself when pressure is applied.

While example embodiments have been described in connection with what is presently considered to be an example of a possible most practical and/or suitable embodiment, it is to be understood that the descriptions are not to be limited to the disclosed embodiments, but on the contrary, is intended to cover various modifications and equivalent arrangements included within the spirit and scope of the example embodiment. Those skilled in the art will recognize or be able to ascertain using no more than routine experimentation, many equivalents to the specific example embodiments specifically described herein. Such equivalents are intended to be encompassed in the scope of the claims, if appended hereto or subsequently filed.

1. A tape which is adherable in the absence of an adhesive layer, is breathable, is reusable and is water-resistant, the tape comprising a fabric layer which includes a first side and a second side and a mixture of a natural wax, a tree resin and a plant oil, the mixture infusing the fabric layer and forming a first outer layer on the first side and a second outer layer on the second side, wherein the tape can be cut to length.

2. The tape of claim 1, wherein the natural wax is beeswax and the plant oil is jojoba oil.

3. The tape of claim 2, wherein the fabric layer is a muslin layer.

4. The tape of claim 3, wherein the mixture is about 35% to about 60% of the weight of the tape.

5. The tape of claim 4, wherein the mixture comprises about 30% to about 50% by weight beeswax, about 9% to about 24% by weight tree resin and about 3% to about 10% by weight jojoba oil.

6. The tape of claim 5, wherein the tree resin is Damar resin.

7. The tape of claim 1, wherein the tape is hand grip tape for use on sporting equipment.

8. A safety kit, the safety kit comprising a tape which is adherable in the absence of an adhesive layer, is anti-microbial, is breathable and is water-resistant, the tape comprising a fabric layer which includes a first side and a second side and a mixture of a natural wax, a tree resin and a plant oil, the mixture infusing the fabric layer and forming a first outer layer on the first side and a second outer layer on the second side, a pair of scissors and a pack of sterile gauze.

9. The safety kit of claim 8, wherein the fabric layer is a muslin layer.

10. The safety kit of claim 9, wherein the mixture is about 35% to about 60% of the weight of the tape.

11. The safety kit of claim 10, wherein the mixture comprises about 30% to about 50% by weight beeswax, about 9% to about 24% by weight tree resin and about 3% to about 10% by weight jojoba oil.

12. The safety kit of claim 11, wherein the tree resin is Damar resin.

13. The safety kit of claim 12, wherein the tape is between about a half inch to about four inches in width and can be cut to length with the scissors.

14. A water-resistant bandage, the water-resistant bandage comprising a tape which is adherable in the absence of an adhesive layer, is anti-microbial, is breathable and is water-resistant, the tape comprising a fabric layer which includes a first side and a second side and a mixture of a natural wax, a tree resin and a plant oil, the mixture infusing the fabric layer and forming a first outer layer on the first side and a second outer layer on the second side, and a gauze which is positioned on the tape such as at least a perimeter of the tape extends beyond the gauze.

15. The water-resistant bandage of claim 14, wherein the fabric layer is a muslin layer.

16. The water-resistant bandage of claim 15, wherein the mixture is about 35% to about 60% of the weight of the tape.

17. The water-resistant bandage of claim 16, wherein the mixture comprises about 30% to about 50% by weight beeswax, about 9% to about 24% by weight tree resin and about 3% to about 10% by weight jojoba oil.

18. The water-resistant bandage of claim 17, wherein the tree resin is Damar resin.

19. The water-resistant bandage of claim 18, wherein the tape is between about a quarter of an inch to about four inches in width.

20. A method of sealing an opening in an article, the method comprising: selecting a tape which is adherable in the absence of an adhesive layer and is water-resistant, the tape comprising a fabric layer which includes a first side and a second side and a mixture of a natural wax, a tree resin and a plant oil, the mixture infusing the fabric layer and forming a first outer layer on the first side and a second outer layer on the second side; cutting the tape to a desired length; and pressing the tape on the article such that the opening is occluded and the tape is wrapped about itself, thereby sealing the opening.
